SEQUENCE LISTING (4 sequences)

SEQ ID NO: 1 - protein, 29 aa, Homo sapiens
MSVLTPLLLRGLTGSARRLPVPRAKIHSL

SEQ ID NO: 2 - DNA, 26 bp, Homo sapiens
attaccggtgcggggttctcatcatc

SEQ ID NO: 3 - DNA, 26 bp, Homo sapiens
gcgcgtaaccttgatacttacctgcg

SEQ ID NO: 4 - DNA, 30 bp, Homo sapiens
attaccggtgcgtcgtaagtggaataagac

(19) United States
(12) Patent Application Publication
(10) Pub. No.: US 2011/0097731 A1
(43) Pub. Date: Apr. 28, 2011

(54) NOVEL MITOCHONDRIAL DYE

(75) Inventors: Thomas Oertner, Riehen (CH); Tobias Rose, Munchen (DE)

(73) Assignee: Novartis Forschungsstiftung, Zweigniederlassung Friedrich Mischer Institute for Biomedical Researc, Basel (CH)

(21) Appl. No.: 12/999,760

(22) PCT Filed: Jun. 24, 2009
(86) PCT No.: PCT/EP2009/057872

§ 371 Date: Dec. 17, 2010

(30) Foreign Application Priority Data
Jun. 25, 2008 (EP) 08158975.6

(51) Int. Cl. C12Q 1/68; C07H 21/04; C12N 15/63; C12N 5/10

(52) U.S. Cl. 435/6; 536/23.1; 435/320.1; 435/325

(57) ABSTRACT

The present invention relates to the use of single-barrel genetically encoded GFP-based calcium indicator as an intramitochondrial dye and to nucleic acid molecules coding for said indicators, as well as to methods using said indicators. Examples of single-barrel genetically encoded GFP-based calcium indicator is a GCaMP, Case16 and/or Case12. In a particular embodiment, the single-barrel genetically encoded GFP-based calcium indicator is GCaMP2 or Case16.

### FIELD OF THE INVENTION

The present invention provides a novel family of molecular mitochondrial dyes.

### BACKGROUND OF THE INVENTION

An increasing number of genetically encoded fluorescent sensors have recently been developed on the basis of GFP-like proteins [1-3]. However, currently available genetically encoded sensors are characterized by low signal intensity and limited dynamic range (maximum change in fluorescence ratio or intensity) [1,4,5], insufficient for routine applications in high throughput screening (HTS) assays and restricting sensitivity of precise single-cell studies. At the same time, genetically encoded sensors provide a much wider flexibility, allowing to be targeted to any chosen cellular compartment, to generate stable cell lines and transgenic animals, to be expressed in a particular tissue and/or in a temporally controlled manner under a specific promoter. Therefore, development of genetically encoded sensors characterized by increased dynamic range and signal intensity remains an actual task. One of the most promising approaches to create genetically encoded sensors is based on the circularly permuted fluorescent protein (cpFP) fused to or inserted into sensitive domain(s) [6-12]. In the presence of an analyte or in response to a cellular event, sensitive domain(s) undergoe(s) structural rearrangements, inducing conformational changes of cpFP and resulting in its altered fluorescent properties. Circular permutation allows placing sensitive domains in a close proximity to the chromophore environment of cpFP within chimeric sensor construct. Therefore, conformational changes of the sensitive domains and their influence on the spectral properties of cpFP is direct and can lead to significant changes in the fluorescent signal.

In particular, such Ca2+ sensors as GCaMPs [8,12] or Pericams [7] were constructed by fusing calmodulin and its target peptide M13 (fragment of myosin light chain kinase) to a cpFP (cpGFP, respectively cpYFP). In the presence of Ca2+, calmodulin binds to the M13 peptide, causing conformational changes in the vicinity of the chromophore and thereby influencing cpFP fluorescence. Similar sensors, named Camgaroos [6,9], are formally based on the non-permuted GFP, but contain an inserted calmodulin molecule at position Tyr145 of EYFP, which is essentially similar to the circular permutation approach. In most cases, it was shown that spectral changes of the cpFP-based sensors fluorescence occur through the chromophore transition from the neutral (protonated) to the charged (anionic) form. Noteworthy, the same mechanism leads to 100-400 fold increase of green fluorescence after photoactivation of so called photoactivatable fluorescent proteins, PA-GFP [13] and PS-CFP [14].

However, despite their increasing number, genetically encoded GFP-based fluorescent sensors were not thought to be really suitable for use within the rather extreme environment, e.g. high pH, of mitochondria. For instance a recent publication (Filippin et al., 2005, Cell Calcium 37:129-136) teaches that only some YFP-based calcium sensors (which have a low dynamic range) are particularly suitable for this purpose, especially since they have a pH sensitivity which is for this purpose considered to be better than that of GFPs. Thus implying that GFPs are not suited to be used a intramitochondrial dyes. Moreover, it was generally believed that single excitation wave-length indicators were less suitable than the excitation ratio indicators such the YFP-based ratiometric PeriCam.

Prior to the present invention there was therefore a need in the art for improved tools which allow understanding the dynamics of calcium within mitochondria.

### SUMMARY OF THE INVENTION

To address this need, the present inventors investigated newly developed genetically encoded fluorescent sensors for their suitability to be used as an intramitochondrial dye.

For example, Souslova et al. (BMC Biotechnology, 2007, 7:37) described the development of high dynamic range cpGFP-based Ca2+ sensors, termed Case12 and Case16, that show up to 16.5-fold increase of the fluorescent signal (F/F0, fluorescence increase, fold) in response to Ca2+. According to Souslova et al., these sensors were more pH stable compared to Flash-pericam [7] and GCaMP1.6 [8] and had been estimated in vitro to have approximately 3-fold higher dynamic range compared to GCaMPs [8,12]. However, despite that advantages highlighted by Souslova et al., Case12 and Case 16 have a lower affinity for Ca2+as compared to standard calcium sensors, as can be seen in Table 2 of said publication of Souslova et al. (BMC Biotechnology, 2007, 7:37).

Despite the “teaching away” presented herein-above, the present inventors investigated the suitability of Case12 and Case16 to be used as an intramitochondrial dye, and surprisingly found that the low affinity of Case12 and Case16 for calcium could be advantageous for the purpose of using them as an intramitochondrial dye. Moreover, the present inventors realized that the low affinity of Case12 and Case16 for calcium, together with its high dynamic range, surprisingly allowed quantitative assays for the concentration of calcium in mitochondria.

Even more surprisingly, the present inventors realized during further investigations that single-barrel genetically encoded GFP-based calcium indicator could in general be used as intramitochondrial dyes, contrary to the opinion prevailing in the art.

The present invention therefore encompasses the use of single-barrel genetically encoded GFP-based calcium indicator as an intramitochondrial dye. Examples of single-barrel genetically encoded GFP-based calcium indicator is a GCaMP, Case16 and/or Case12. In a particular embodiment, the single-barrel genetically encoded GFP-based calcium indicator is GCaMP2 or Case16.

The present invention also encompasses an isolated nucleic acid comprising (i) a nucleotide sequence encoding for a single-barrel genetically encoded GFP-based calcium indicator functionally linked to a mitochondrial targeting signal, or (ii) the nucleotide sequence complementary to the nucleotide sequence of (i). In a particular embodiment, the isolated nucleic acid of the invention uses the mitochondrial targeting signal MSVLTPLLLRGLTGSARRLPVPRAKIHSL (SEQ ID NO:1). In some embodiments of the invention, the isolated nucleic acid further comprises a promoter capable of inducing the expression of the product encoded by said nucleic acid in neurons, for instance the neuron-specific synapsin-I (syn) promoter. The single-barrel genetically encoded GFP-based calcium indicator present in the isolated nucleic acid of the invention can be a GCaMP, Case16 and/or Case12. In some embodiments, the single-barrel genetically encoded GFP-based calcium indicator is GCaMP2 or Case16.

The present invention also encompasses a recombinant vector comprising the isolated nucleic acid of the invention and/or a host cell comprising said vector.

A further aspect of the invention is a kit comprising an isolated nucleic according to the invention, a recombinant vector according to the invention or a host cell according to the invention.

Yet a further aspect of the invention is a method of assaying the function and/or viability of mitochondria comprising the steps of expressing an isolated nucleic according to the invention, or a recombinant vector according to the invention, in a cell and, of assessing any calcium-dependent shift of fluorescence in the mitochondria of said cell.

A further method of the invention is a method of screening for agents affecting the function and/or viability of mitochondria comprising the steps of expressing an isolated nucleic according to the invention, or a recombinant vector according to the invention, in a cell and of assessing any calcium-dependent shift of fluorescence in the mitochondria of said cell in the presence of said agent as compared to the fluorescence in the mitochondria measured in the absence of said agent.

In some of the embodiments of the methods of the invention, fluorescence is measured by laser scanning microscopy, e.g. 2-photon microscopy or confocal microscopy.

Moreover, in some embodiments of the invention described herein, the single-barrel genetically encoded GFP-based calcium indicator is based on circularly permuted GFP (cpGFP).

### DESCRIPTION OF THE FIGURES

FIG. 1: The mitochondrial localization efficiency of the GFP-based single-barrel mitochondrial Ca2+indicators mCase16 and mGCaMP2 is superior to double-barrel indicators (mt8YellowChameleon is a representative example) and better than the most recent version of the YFP-based single-barrel mitochondrial Ca2+ indicator 2mt8RatiometricPericam. White bars indicate data from (Filippin et al., 2005); black bars show own data. (mCase16: 8 cells, mGCaMP2: 9 cells; data±S.E.M.). The inventors performed two-photon imaging of transfected CA3 pyramidal neurons in hippocampal brain slices. Regions of interest (ROIs) were selected covering mitochondria or nuclear regions.

FIG. 2: Pharmacological dissipation of the mitochondrial membrane potential demonstrates that mGCaMP2 exclusively reports mitochondrial Ca²⁺ elevation. Neurons in brain tissue slice culture were transfected with mGCaMP2 and trains of action potentials were evoked by somatic current injection in the whole-cell patch clamp configuration (A & B, lower gray traces). The resulting Ca²⁺ influx into the cytosol leads to mitochondrial Ca²⁺ uptake that can readily be detected by two-photon microscopy as an increase in the somatic mGCaMP2 fluorescence (A, solid black trace a). Mitochondrial Ca²⁺ uptake relies on the membrane potential gradient across the inner mitochondrial membrane generated by the mitochondrial proton transporters. Dissipation of this gradient by addition of the protonophore Carbonyl cyanide 3-chlorophenylhydrazone (CCCP) therefore reversible inhibits mitochondrial Ca²⁺ uptake while leaving cytosolic Ca²⁺ unaffected. This effect can be monitored with mGCaMP2 as a complete loss of the fluorescence response to stimulation 15 min after addition of CCCP (A, dotted black trace b; B, trace b) and the complete recovery after washout of the agent (A, dashed black trace c; B, trace c). Mistargeted cytosolic mGCaMP2 would still report cytosolic increases in Ca²⁺ as has been demonstrated for mitochondrial ratiometric pericam (Filippin et al., 2005). The complete absence of a fluorescence change therefore further demonstrates the superior targeting of mGCaMP2 to the mitochondrial matrix.

FIG. 3: mGCaMP2 is a high-affinity indicator for mitochondrial Ca²⁺ whereas mCase16 has a lower affinity but a higher dynamic range. Both sensors therefore can be used complementary to assay a broader range of mitochondrial Ca²⁺ changes. Neurons in brain tissue slice culture were transfected with mGCaMP2 and mCase16, respectively, and trains of action potentials of varying frequency and action potential number were evoked by somatic current injection. Whereas with mGCaMP2 a clear increase in mitochondrial Ca²⁺ can be detected at the lowest stimulation intensity, the cell expressing mCase16 did not show a fluorescence change (A & B, dotted traces). With stronger stimulation also mCase16 showed a robust response that, in contrast to mGCaMP2, did not saturate (A & B, dashed and solid traces). This indicates that mGCaMP2 has a higher Ca²⁺ affinity than mCase16. Furthermore, mCase16 showed a more pronounced total fluorescence change at the strongest stimulation intensity and therefore has a higher dynamic range than mGCaMP2.

FIG. 4: mGCaMP2 allows the detection of robust fluorescence signals in individual small mitochondria (<0.5 μm) in response to mitochondrial Ca²⁺ elevations. A subtype of presynaptic structures (Schaffer Collateral boutons) in hippocampal brain tissue predominantly contains individual mitochondria (Shepherd and Harris, 1998). Boutons of this type were imaged by two-photon microscopy of mGCaMP2-expressing cells in brain tissue slice culture. Ca²⁺ influx into boutons was evoked by eliciting trains of action potentials at the soma of these cells (A&B, lower panels). This leads to the surprisingly fast accumulation of Ca²⁺ in individual mitochondria that can readily be detected as robust fluorescence increase around 520 nm (A&B, upper panels). Signals can be obtained at both low (A, frame scanning mode) and high (B, line scanning mode) temporal resolution. Examples shown are individual traces from two different mitochondria in boutons of two different cells.

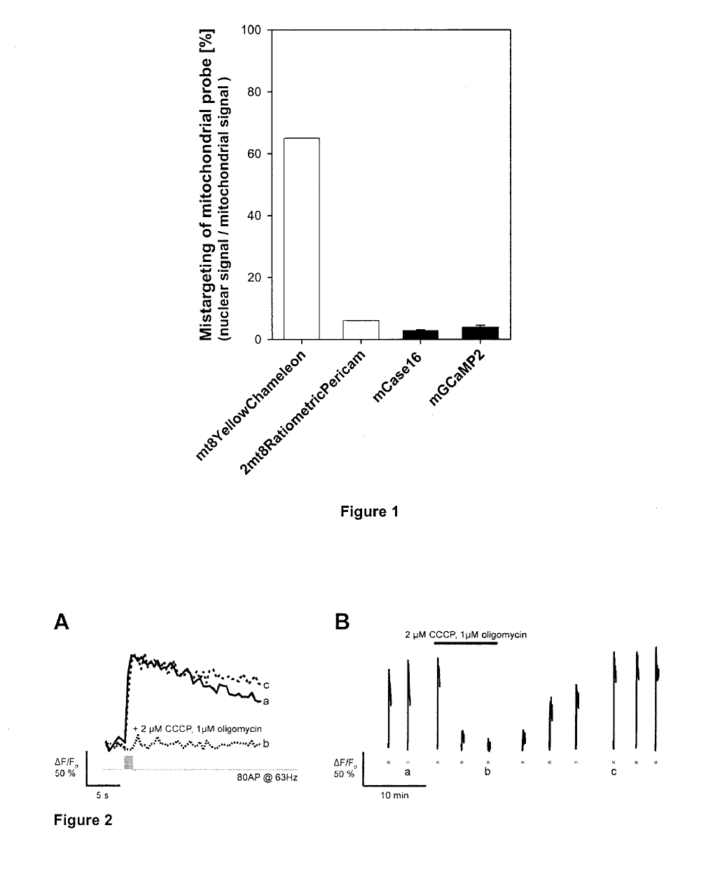

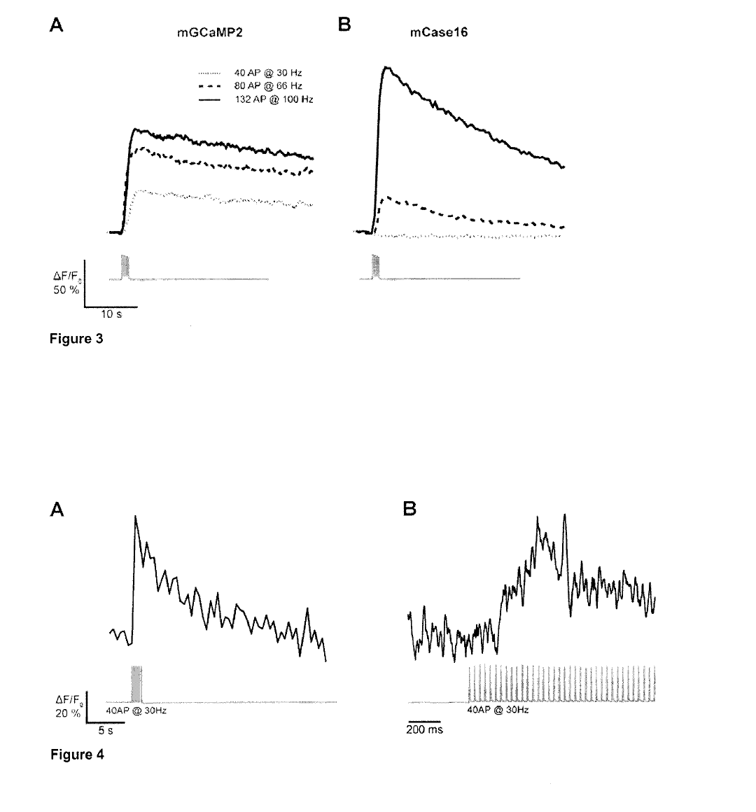

### DETAILED DESCRIPTION OF THE INVENTION

As explained herein-above, there was a desire in the art for means allowing to measure intramitochondrial calcium ceontent/concentration. To address this need, the present inventors investigated newly developed genetically encoded fluorescent sensors for their suitability to be used as an intramitochondrial dye.

For example, Souslova et al. (BMC Biotechnology, 2007, 7:37) described the development of high dynamic range cpGFP-based Ca2+ sensors, termed Case12 and Case16, that show up to 16.5-fold increase of the fluorescent signal (F/F0, fluorescence increase, fold) in response to Ca2+. According to Souslova et al., these sensors were more pH stable compared to Flash-pericam [7] and GCaMP1.6 [8] and had been estimated in vitro to have approximately 3-fold higher dynamic range compared to GCaMPs [8,12]. However, despite that advantages highlighted by Souslova et al., Case12 and Case16 have a lower affinity for Ca2+ as compared to standard calcium sensors, as can be seen in Table 2 of said publication of Souslova et al. (BMC Biotechnology, 2007, 7:37).

Despite the “teaching away” presented herein-above, the present inventors investigated the suitability of Case12 and Case16 to be used as an intramitochondrial dye, and surprisingly found that the low affinity of Case12 and Case16 for calcium could be advantageous for the purpose of using them as an intramitochondrial dye. Moreover, the present inventors realized that the low affinity of Case12 and Case16 for calcium, together with its high dynamic range, surprisingly allowed quantitative assays for the concentration of calcium in mitochondria.

Even more surprisingly, the present inventors realized during further investigations that single-barrel genetically encoded GFP-based calcium indicator could in general be used as intramitochondrial dyes, contrary to the opinion prevailing in the art.

The present invention therefore encompasses the use of single-barrel genetically encoded GFP-based calcium indicator as an intramitochondrial dye. Examples of single-barrel genetically encoded GFP-based calcium indicator is a GCaMP, Case16 and/or Case12. In a particular embodiment, the single-barrel genetically encoded GFP-based calcium indicator is GCaMP2 or Case16.

The present invention also encompasses an isolated nucleic acid comprising (i) a nucleotide sequence encoding for a single-barrel genetically encoded GFP-based calcium indicator functionally linked to a mitochondrial targeting signal, or (ii) the nucleotide sequence complementary to the nucleotide sequence of (i). In a particular embodiment, the isolated nucleic acid of the invention uses the mitochondrial targeting signal MSVLTPLLLRGLTGSARRLPVPRAKIHSL (SEQ ID NO:1). In some embodiments of the invention, the isolated nucleic acid further comprises a promoter capable of inducing the expression of the product encoded by said nucleic acid in neurons, for instance the neuron-specific synapsin-I (syn) promoter. The single-barrel genetically encoded GFP-based calcium indicator present in the isolated nucleic acid of the invention can be a GCaMP, Case16 and/or Case12. In some embodiments, the single-barrel genetically encoded GFP-based calcium indicator is GCaMP2 or Case16.

The present invention also encompasses a recombinant vector comprising the isolated nucleic acid of the invention and/or a host cell comprising said vector.

A further aspect of the invention is a kit comprising an isolated nucleic according to the invention, a recombinant vector according to the invention or a host cell according to the invention.

Yet a further aspect of the invention is a method of assaying the function and/or viability of mitochondria comprising the steps of expressing an isolated nucleic according to the invention, or a recombinant vector according to the invention, in a cell and, of assessing any calcium-dependent shift of fluorescence in the mitochondria of said cell.

A further method of the invention is a method of screening for agents affecting the function and/or viability of mitochondria comprising the steps of expressing an isolated nucleic according to the invention, or a recombinant vector according to the invention, in a cell and of assessing any calcium-dependent shift of fluorescence in the mitochondria of said cell in the presence of said agent as compared to the fluorescence in the mitochondria measured in the absence of said agent. The inventions is also meant to encompass the agents affecting the function and/or viability of mitochondria identified using the methods of the invention.

In some of the embodiments of the methods of the invention, fluorescence is measured by laser scanning microscopy, e.g. 2-photon microscopy or confocal microscopy.

Moreover, in some embodiments of the invention described herein, the single-barrel genetically encoded GFP-based calcium indicator is based on circularly permuted GFP (cpGFP).

In addition, in some of the embodiments of the invention, the single-barrel genetically encoded GFP-based calcium indicator is a single-wave length GFP-based calcium indicator.

These and other aspects of the present invention should be apparent to those skilled in the art, from the teachings herein.

For convenience, the meaning of certain terms and phrases employed in the specification, examples, and appended claims are provided below.

The singular forms “a, “”an,” and “the” include plural reference unless the context clearly dictates otherwise.

A “single-barrel genetically encoded GFP-based calcium indicator” is a non-FRET, single wavelength, GFP-based indicator. Examples thereof are G-CaMP, e.g. GCaMP2, Case-12 and Case-16.

“Polynucleotide” and “nucleic acid”, used interchangeably herein, refer to polymeric forms of nucleotides of any length, either ribonucleotides or deoxyribonucleotides. Thus, these terms include, but are not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. These terms further include, but are not limited to, mRNA or cDNA that comprise intronic sequences. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidites and thus can be an oligodeoxynucleoside phosphoramidate or a mixed phosphoramidate-phosphodiester oligomer. A polynucleotide may comprise modified nucleotides, such as methylated nucleotides and nucleotide analogs, uracyl, other sugars, and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support. The term “polynucleotide” also encompasses peptidic nucleic acids, PNA and LNA. Polynucleotides may further comprise genomic DNA, cDNA, or DNA-RNA hybrids.

“Sequence Identity” refers to a degree of similarity or complementarity. There may be partial identity or complete identity. A partially complementary sequence is one that at least partially inhibits an identical sequence from hybridizing to a target polynucleotide; it is referred to using the functional term “substantially identical.” The inhibition of hybridization of the completely complementary sequence to the target sequence may be examined using a hybridization assay (Southern or Northern blot, solution hybridization and the like) under conditions of low stringency. A substantially identical sequence or probe will compete for and inhibit the binding (i.e., the hybridization) of a completely identical sequence or probe to the target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarities (e.g., less than about 30% identity); in the absence of non-specific binding, the probe will not hybridize to the second non-complementary target sequence.

Another way of viewing sequence identity in the context to two nucleic acid or polypeptide sequences includes reference to residues in the two sequences that are the same when aligned for maximum correspondence over a specified region. As used herein, percentage of sequence identity means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison and multiplying the result by 100 to yield the percentage of sequence identity.

“Gene” refers to a polynucleotide sequence that comprises control and coding sequences necessary for the production of a polypeptide or precursor. The polypeptide can be encoded by a full length coding sequence or by any portion of the coding sequence. A gene may constitute an uninterrupted coding sequence or it may include one or more introns, bound by the appropriate splice junctions. Moreover, a gene may contain one or more modifications in either the coding or the untranslated regions that could affect the biological activity or the chemical structure of the expression product, the rate of expression, or the manner of expression control. Such modifications include, but are not limited to, mutations, insertions, deletions, and substitutions of one or more nucleotides. In this regard, such modified genes may be referred to as “variants” of the “native” gene.

“Expression” generally refers to the process by which a polynucleotide sequence undergoes successful transcription and translation such that detectable levels of the amino acid sequence or protein are expressed. In certain contexts herein, expression refers to the production of mRNA. In other contexts, expression refers to the production of protein.

“Cell type” refers to a cell from a given source (e.g., tissue or organ) or a cell in a given state of differentiation, or a cell associated with a given pathology or genetic makeup.

“Polypeptide” and “protein”, used interchangeably herein, refer to a polymeric form of amino acids of any length, which may include translated, untranslated, chemically modified, biochemically modified, and derivatized amino acids. A polypeptide or protein may be naturally occurring, recombinant, or synthetic, or any combination of these. Moreover, a polypeptide or protein may comprise a fragment of a naturally occurring protein or peptide. A polypeptide or protein may be a single molecule or may be a multi-molecular complex. In addition, such polypeptides or proteins may have modified peptide backbones. The terms include fusion proteins, including fusion proteins with a heterologous amino acid sequence, fusions with heterologous and homologous leader sequences, with or without N-terminal methionine residues, immunologically tagged proteins, and the like.

A “fragment of a protein” refers to a protein that is a portion of another protein. For example, fragments of proteins may comprise polypeptides obtained by digesting full-length protein isolated from cultured cells. In one embodiment, a protein fragment comprises at least about 6 amino acids. In another embodiment, the fragment comprises at least about 10 amino acids. In yet another embodiment, the protein fragment comprises at least about 16 amino acids.

An “expression produce” or “gene product” is a biomolecule, such as a protein or mRNA, that is produced when a gene in an organism is transcribed or translated or post-translationally modified.

“Host cell” refers to a microorganism, a prokaryotic cell, a eukaryotic cell or cell line cultured as a unicellular entity that may be, or has been, used as a recipient for a recombinant vector or other transfer of polynucleotides, and includes the progeny of the original cell that has been transfected. The progeny of a single cell may not necessarily be completely identical in morphology or in genomic or total DNA complement as the original parent due to natural, accidental, or deliberate mutation.

The term “functional equivalent” is intended to include the “fragments”, “mutants”, “derivatives”, “alleles”, “hybrids”, “variants”, “analogs”, or “chemical derivatives” of the native gene or virus.

“Isolated” refers to a polynucleotide, a polypeptide, an immunoglobulin, a virus or a host cell that is in an environment different from that in which the polynucleotide, the polypeptide, the immunoglobulin, the virus or the host cell naturally occurs.

“Substantially purified” refers to a compound that is removed from its natural environment and is at least about 60% free, at least about 65% free, at least about 70% free, at least about 75% free, at least about 80% free, at least about 83% free, at least about 85% free, at least about 88% free, at least about 90% free, at least about 91% free, at least about 92% free, at least about 93% free, at least about 94% free, at least about 95% free, at least about 96% free, at least about 97% free, at least about 98% free, at least about 99% free, at least about 99.9% free, or at least about 99.99% or more free from other components with which it is naturally associated.

“Diagnosis” and “diagnosing” generally includes a determination of a subject's susceptibility to a disease or disorder, a determination as to whether a subject is presently affected by a disease or disorder, a prognosis of a subject affected by a disease or disorder (e.g., identification of pre-metastatic or metastatic cancerous states, stages of cancer, or responsiveness of cancer to therapy), and therametrics (e.g., monitoring a subject's condition to provide information as to the effect or efficacy of therapy).

“Biological sample” encompasses a variety of sample types obtained from an organism that may be used in a diagnostic or monitoring assay. The term encompasses blood and other liquid samples of biological origin, solid tissue samples, such as a biopsy specimen, or tissue cultures or cells derived therefrom and the progeny thereof. The term specifically encompasses a clinical sample, and further includes cells in cell culture, cell supernatants, cell lysates, serum, plasma, urine, amniotic fluid, biological fluids, and tissue samples. The term also encompasses samples that have been manipulated in any way after procurement, such as treatment with reagents, solubilization, or enrichment for certain components.

“Individual”, “subject”, “host” and “patient”, used interchangeably herein, refer to any mammalian subject for whom diagnosis, treatment, or therapy is desired. In one preferred embodiment, the individual, subject, host, or patient is a human. Other subjects may include, but are not limited to, cattle, horses, dogs, cats, guinea pigs, rabbits, rats, primates, and mice.

“Hybridization” refers to any process by which a polynucleotide sequence binds to a complementary sequence through base pairing. Hybridization conditions can be defined by, for example, the concentrations of salt or formamide in the prehybridization and hybridization solutions, or by the hybridization temperature, and are well known in the art. Hybridization can occur under conditions of various stringency.

“Stringent conditions” refers to conditions under which a probe may hybridize to its target polynucleotide sequence, but to no other sequences. Stringent conditions are sequence-dependent (e. g., longer sequences hybridize specifically at higher temperatures). Generally, stringent conditions are selected to be about 5° C. lower than the thermal melting point (Tm) for the specific sequence at a defined ionic strength and pH. The Tm is the temperature (under defined ionic strength, pH, and polynucleotide concentration) at which 50% of the probes complementary to the target sequence hybridize to the target sequence at equilibrium. Typically, stringent conditions will be those in which the salt concentration is at least about 0.01 to about 1.0 M sodium ion concentration (or other salts) at about pH 7.0 to about pH 8.3 and the temperature is at least about 30° C. for short probes (e. g., 10 to 50 nucleotides).

Stringent conditions may also be achieved with the addition of destabilizing agents, such as formamide.

“Biomolecule” includes polynucleotides and polypeptides.

“Biological activity” refers to the biological behavior and effects of a protein or peptide. The biological activity of a protein may be affected at the cellular level and the molecular level. For example, the biological activity of a protein may be affected by changes at the molecular level. For example, an antisense oligonucleotide may prevent translation of a particular mRNA, thereby inhibiting the biological activity of the protein encoded by the mRNA. In addition, an immunoglobulin may bind to a particular protein and inhibit that protein's biological activity.

“Oligonucleotide” refers to a polynucleotide sequence comprising, for example, from about 10 nucleotides (nt) to about 1000 nt. Oligonucleotides for use in the invention are for instance from about 15 nt to about 150 nt, for instance from about 150 nt to about 1000 nt in length. The oligonucleotide may be a naturally occurring oligonucleotide or a synthetic oligonucleotide.

“Modified oligonucleotide” and “Modified polynucleotide” refer to oligonucleotides or polynucleotides with one or more chemical modifications at the molecular level of the natural molecular structures of all or any of the bases, sugar moieties, internucleoside phosphate linkages, as well as to molecules having added substitutions or a combination of modifications at these sites. The internucleoside phosphate linkages may be phosphodiester, phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone internucleotide linkages, or 3′-3′, 5′-3′, or 5′-5′ linkages, and combinations of such similar linkages. The phosphodiester linkage may be replaced with a substitute linkage, such as phosphorothioate, methylamino, methylphosphonate, phosphoramidate, and guanidine, and the ribose subunit of the polynucleotides may also be substituted (e. g., hexose phosphodiester; peptide nucleic acids). The modifications may be internal (single or repeated) or at the end (s) of the oligonucleotide molecule, and may include additions to the molecule of the internucleoside phosphate linkages, such as deoxyribose and phosphate modifications which cleave or crosslink to the opposite chains or to associated enzymes or other proteins. The terms “modified oligonucleotides” and “modified polynucleotides” also include oligonucleotides or polynucleotides comprising modifications to the sugar moieties (e. g., 3′-substituted ribonucleotides or deoxyribonucleotide monomers), any of which are bound together via 5′to 3′linkages.

“Biomolecular sequence” or “sequence” refers to all or a portion of a polynucleotide or polypeptide sequence.

The term “detectable” refers to a polynucleotide expression pattern which is detectable via the standard techniques of polymerase chain reaction (PCR), reverse transcriptase—(RT) PCR, differential display, and Northern analyses, which are well known to those of skill in the art. Similarly, polypeptide expression patterns may be “detected” via standard techniques including immunoassays such as Western blots.

A “target gene” refers to a polynucleotide, often derived from a biological sample, to which an oligonucleotide probe is designed to specifically hybridize. It is either the presence or absence of the target polynucleotide that is to be detected, or the amount of the target polynucleotide that is to be quantified. The target polynucleotide has a sequence that is complementary to the polynucleotide sequence of the corresponding probe directed to the target. The target polynucleotide may also refer to the specific subsequence of a larger polynucleotide to which the probe is directed or to the overall sequence (e.g., gene or mRNA) whose expression level it is desired to detect.

A “target protein” refers to a polypeptide, often derived from a biological sample, to which a protein-capture agent specifically hybridizes or binds. It is either the presence or absence of the target protein that is to be detected, or the amount of the target protein that is to be quantified. The target protein has a structure that is recognized by the corresponding protein-capture agent directed to the target. The target protein or amino acid may also refer to the specific substructure of a larger protein to which the protein-capture agent is directed or to the overall structure (e. g., gene or mRNA) whose expression level it is desired to detect.

“Complementary” refers to the topological compatibility or matching together of the interacting surfaces of a probe molecule and its target. The target and its probe can be described as complementary, and furthermore, the contact surface characteristics are complementary to each other. Hybridization or base pairing between nucleotides or nucleic acids, such as, for example, between the two strands of a double-stranded DNA molecule or between an oligonucleotide probe and a target are complementary.

“Label” refers to agents that are capable of providing a detectable signal, either directly or through interaction with one or more additional members of a signal producing system. Labels that are directly detectable and may find use in the invention include fluorescent labels. Specific fluorophores include fluorescein, rhodamine, BODIPY, cyanine dyes and the like.

The term “fusion protein” refers to a protein composed of two or more polypeptides that, although typically not joined in their native state, are joined by their respective amino and carboxyl termini through a peptide linkage to form a single continuous polypeptide. It is understood that the two or more polypeptide components can either be directly joined or indirectly joined through a peptide linker/spacer.

The term “normal physiological conditions” means conditions that are typical inside a living organism or a cell. Although some organs or organisms provide extreme conditions, the intra-organismal and intra-cellular environment normally varies around pH 7 (i.e., from pH 6.5 to pH 7.5), contains water as the predominant solvent, and exists at a temperature above 0° C. and below 50° C. The concentration of various salts depends on the organ, organism, cell, or cellular compartment used as a reference.

“BLAST” refers to Basic Local Alignment Search Tool, a technique for detecting ungapped sub-sequences that match a given query sequence.

“BLASTP” is a BLAST program that compares an amino acid query sequence against a protein sequence database. “BLASTX” is a BLAST program that compares the six-frame conceptual translation products of a nucleotide query sequence (both strands) against a protein sequence database.

A “cds” is used in a GenBank DNA sequence entry to refer to the coding sequence. A coding sequence is a sub-sequence of a DNA sequence that is surmised to encode a gene.

A “consensus” or “contig sequence”, as understood herein, is a group of assembled overlapping sequences, particularly between sequences in one or more of the databases of the invention.

The sensors as used in the present invention can be produced by a virus harbouring a nucleic acid that encodes the sensor gene sequence. The virus may comprise elements capable of controlling and/or enhancing expression of the nucleic acid. The virus may be a recombinant virus. The recombinant virus may also include other functional elements. For instance, recombinant viruses can be designed such that the viruses will autonomously replicate in the target cell. In this case, elements that induce nucleic acid replication may be required in a recombinant virus. The recombinant virus may also comprise a promoter or regulator or enhancer to control expression of the nucleic acid as required. Tissue specific promoter/enhancer elements may be used to regulate expression of the nucleic acid in specific cell types. The promoter may be constitutive or inducible.

Contaminant components of its natural environment are materials that would interfere with the methods and compositions of the invention, and may include enzymes, hormones, and other proteinaceous or nonproteinaceous solutes. Ordinarily, an isolated agent will be prepared by at least one purification step. In one embodiment, the agent is purified to at least about 60%, at least about 65%, at least about 70%, at least about 75%, at least about 80%, at least about 85%, at least about 88%, at least about 90%, at least about 92%, at least about 95%, at least about 97%, at least about 98%, at least about 99%, at least about 99.9%, or at least about 99.99% by weight of e.g. virus.

A “test agent” refers to any molecule, material, or treatment that is tested in a screen. The molecule may be randomly selected for inclusion in the screen, or may be included because of an a priori expectation that the molecule will give a positive result in the screen. Molecules can include any known chemical or biochemical molecule, including peptides, nucleic acids, carbohydrates, lipids, or any other organic or inorganic molecule. A “test agent” can also refer to non-molecular entities, such as electromagnetic radiation or heat. It will be appreciated by those of skill in the art that there are many commercial suppliers of chemical compounds, including Sigma Chemical Co. (St. Louis, Mo.), Aldrich Chemical Co. (St. Louis, Mo.), Sigma-Aldrich (St. Louis, Mo.), Fluka Chemika-Biochemica Analytika (Buchs Switzerland), and the like. Essentially any chemical compound can be used as a potential activity modulator in the assays of the invention, although most often compounds that can be dissolved in aqueous or organic (especially DMSO-based) solutions are used. In some embodiments, high-throughput screening methods can involve providing a combinatorial library containing a large number of potential therapeutic compounds (potential modulator compounds). Such “combinatorial chemical libraries” are then screened in one or more assays to identify those library members (particular chemical species or subclasses) that display a desired characteristic activity. The compounds thus identified can serve as conventional “lead compounds” or can themselves be used as potential or actual therapeutics. A combinatorial chemical library is a collection of diverse chemical compounds generated by either chemical synthesis or biological synthesis, by combining a number of chemical“building blocks,”such as agents. For example, a linear combinatorial chemical library, such as a polypeptide library, is formed by combining a set of chemical building blocks (e. g., amino acids) in every possible way for a given compound length (i. e., the number of amino acids in a polypeptide compound). Millions of chemical compounds can be synthesized through such combinatorial mixing of chemical building blocks. Preparation and screening of combinatorial chemical libraries is well known to those of skill in the art. Such combinatorial chemical libraries include, but are not limited to, peptide libraries (see, e. g., U.S. Pat. No. 5,010,175, Furka, Int. J. Pept. Prot. Res., 37: 487-493 (1991) and Houghton, et al., Nature, 354: 84-88 (1991)). Other chemistries for generating chemical diversity libraries can also be used. Such chemistries include, but are not limited to, peptoids (PCT Publication No. WO 91/19735); encoded peptides (PCT Publication WO 93/20242); random bio-oligomers (PCT Publication No. WO 92/00091); benzodiazepines (U.S. Pat. No. diversomers, such as hydantoins, benzodiazepines and dipeptides (Hobbs, et al., Proc. Nat. Acad. Sci. US. 4, 90: 6909-6913 (1993)); vinylogous polypeptides (Hagihara, et al., J. Amer. Chem. Soc. 114: 6568 (1992)); nonpeptidal peptidomimetics with (3-D-glucose scaffolding (Hirschmann, et al., J. Amer. Chem. Soc., 114: 9217-9218 (1992)); analogous organic syntheses of small compound libraries (Chen, et al., J. Amer. Chem. Soc., 116: 2661 (1994)); oligocarbamates (Cho, et al., Science, 261: 1303 (1993)); and/or peptidyl phosphonates (Campbell, et al., J. Org. Chem. 59: 658 (1994)); nucleic acid libraries (see, Ausubel, Berger and Sambrook, all supra); peptide nucleic acid libraries (see, e. g., U.S. Pat. No. 5,539,083); antibody libraries (see, e. g., Vaughn, et al., Nature Biotechnology, 14 (3): 309-314 (1996) and PCT/US96/10287); carbohydrate libraries (see, e. g., Liang, et al., Science, 274: 1520-1522 (1996) and U.S. Pat. No. 5,593,853); small organic molecule libraries (see, e. g., benzodiazepines, Baum C&E News, Jan. 18, page 33 (1993); isoprenoids (U.S. Pat. No. 5,569,588); thiazolidinones and metathiazanones (U.S. Pat. No. 5,549,974); pyrrolidines (U.S. Pat. Nos. 5,525,735 and 5,519,134); morpholino compounds (U.S. Pat. No. 5,506,337); benzodiazepines (U.S. Pat. No. and the like. Devices for the preparation of combinatorial libraries are commercially available (see, e. g., 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e. g., ComGenex, Princeton, N.J., Asinex, Moscow, Russia, Tripos, Inc., St. Louis, Mo., ChemStar, Ltd., Moscow, RU, 3D Pharmaceuticals, Exton, Pa., Martek Biosciences, Columbia, Md., etc.).

When a test agent is said to “modulate” an activity or the calcium content of mitochondria, this means that said activity or said calcium content of the mitochondria is detectably altered. In certain embodiments, a “modulation” can be detected as a difference in, e. g., fluorescence intensity or in a translocation of a detectable signal. In some embodiments, such fluorescence will be measurable, and a “modulation” will comprise a statistically significant alteration in the fluorescence. However, a “modulation” can also refer to detection of a change by any means, such as a subjective determination by a human observer.

An “uncoupling protein” refers to any polypeptide that acts to alter the mitochondrial membrane potential in a cell, e. g., that dissipates the mitochondrial membrane potential. Uncoupling proteins include, but are not limited to, UCP 1 (or “UCP” see e.g. Cassard et al., (1990) J Cell Biochem 43: 255-64; see also GenBank Accession No. U28480); UCP2 (see, e. g., Fleury et al., (1997) Nattere Genet. 15: 269-272; see, also, GenBank Accession No. AF096289), UCP3 (see, e. g., Boss et al., (1997) FEBSLett. 408: 39-42; see, also, GenBank Accession No. NM 003356), UCP4 (see, e. g., Mao et al., (1999) FEBSLett. 443: 326-30; see, also, GenBank Accession No. AF110532), and BMCP1 (see, e. g., Sanchis et al., (1998) J. Biol. Chem. 273: 34611-5; see, also, GenBank Accession No. AF078544), or any homolog, variant, fragment, or derivative thereof, from any source including humans. The ability of a polypeptide to alter mitochondrial membrane potential can be assessed numerous methods well-known to the skilled person.

“Expressing” a protein in a cell means to ensure that the protein is present in the cell, e. g., for the purposes of a procedure of interest. In numerous embodiments, “expressing” a protein will comprise introducing a transgene into a cell comprising a polynucleotide encoding the protein, operably linked to a promoter, wherein the promoter is a constitutive promoter, or an inducible promoter where the conditions sufficient for induction are created, as well as a localization sequence. However, a cell that, e. g., naturally expresses a protein of interest, can be used without manipulation and is considered as “expressing” the protein.

A “fluorescent probe” refers to any compound with the ability to emit light of a certain wavelength when activated by light of another wavelength.

“Fluorescence” refers to any detectable characteristic of a fluorescent signal, including intensity, spectrum, wavelength, intracellular distribution, etc.

“Membrane potential” refers to a difference in the electrical potential across a membrane such as a mitochondrial membrane. In the context of the present invention, such differences reflect transmembrane differences in the concentrations of charged molecules, such as sodium, potassium, and, particularly in the case of mitochondrial membranes, protons and calcium.

“Detecting” fluorescence refers to assessing the fluorescence of a cell using qualitative or quantitative methods. For instance, the fluorescence is determined using quantitative means, e. g., measuring the fluorescence intensity, spectrum, or intracellular distribution, allowing the statistical comparison of values obtained under different conditions. The level can also be determined using qualitative methods, such as the visual analysis and comparison by a human of multiple samples, e. g., samples detected using a fluorescent microscope or other optical detector (e. g., image analysis system, etc.) An “alteration” or “modulation” in fluorescence refers to any detectable difference in the intensity, intracellular distribution, spectrum, wavelength, or other aspect of fluorescence under a particular condition as compared to another condition. For example, an “alteration” or “modulation” is detected quantitatively, and the difference is a statistically significant difference. Any “alterations” or “modulations” in fluorescence can be detected using standard instrumentation, such as a fluorescent microscope, CCD, or any other fluorescent detector, and can be detected using an automated system, such as the integrated systems, or can reflect a subjective detection of an alteration by a human observer.

An assay performed in a “homogeneous format” means that the assay can be performed in a single container, with no manipulation or purification of any components being required to determine the result of the assay, e. g., a test agent can be added to an assay system and any effects directly measured. Often, such “homogeneous format” assays will comprise at least one component that is “quenched” or otherwise modified in the presence or absence of a test agent. For example, in classical assays fluorescent dyes can be present within the mitochondrial matrix in the absence of uncoupling activity, and the fluorescence is quenched. In the presence of uncoupling activity, however, the dyes move to the extramitochondrial space, thereby reducing the level of quenching of the dye, and increasing the fluorescent signal in the cell.

A “secondary screening step” refers to a screening step whereby a test agent is assessed for a secondary property in order to determine the specificity or mode of action of a compound identified using the methods provided herein. Such secondary screening steps can be performed on all of the test agents, or, e. g., on only those that are found to be positive in a primary screening step, and can be performed subsequently, simultaneously, or prior to a primary screening step.

“High-throughput screening” refers to a method of rapidly assessing a large number of test agents for a specific activity. Typically, the plurality of test agents will be assessed in parallel, for example by simultaneously assessing 96 or 384 agents using a 96-well or 384-well plate, 96-well or 384-well dispensers, and detection methods capable of detecting 96 or 384 samples simultaneously. Often, such methods will be automated, e. g., using robotics.

“Robotic high-throughput screening” refers to high-throughput screening that involves at least one robotic element, thereby eliminating a requirement for human manipulation in at least one step of the screening process. For example, a robotic arm can dispense a plurality of test agents to a multi-well plate.

A “multi-well plate” refers to any container, receptacle, or device that can hold a plurality of samples, e. g., for use in high-throughput screening. Typically, such “multi-well plates” will be part of an integrated and preferably automated system that enables the rapid and efficient screening or manipulation of a large number of samples. Such plates can include, e. g., 24, 48, 96, 384, 768, 1536, or more wells, and are typically used in conjunction with a 24, 48, 96, 384, 768, 1536, or more tip pipettors, samplers, detectors, etc.

A “permeabilizing agent” refers to any agent that acts to permeabilize the wall of a cell or of a cellular compartment. Such agents may comprise enzymes that act to degrade the cell wall, such as zymolyase or chitinase, or can comprise chemical agents that can permeabilize the wall by chemical means.

Any of a number of cell types can be used in the present invention. For example, any eukaryotic cell, including plant, animal, and fungal cells can be used. In some embodiments, neurone will be used. As used herein, “cells” can include whole cells (untreated cells), permeabilized cells, isolated mitochondria, and proteoliposomes, e. g., proteoliposomes reconstituted with a UCP or another protein of interest. The care and maintenance of cells, including yeast cells, is well known to those of skill in the art and can be found in any of a variety of sources, such as Freshney (1994) Culture of Animal Cells. Manual of Basic Technique, Wiley-Liss, New York, Guthrie & Fink (1991), Guthrie and Fink, Guide to Yeast Genetics and Molecular Biology, Academic Press, Ausubel et al. (1999) Current Protocols in Molecular Biology, Greene Publishing Associates, and others.

In some embodiments, mammalian, insect, or other metazoan cells can be used to test for agents that are capable of inducing apoptosis or that otherwise affect mitochondrial membrane potential. Any such cell type can be used, including primary cell lines, secondary cell lines, transformed cells, and others, and including whole (untreated) cells, permeabilized cells, isolated mitochondria, and proteoliposomes. For example, a number of cell types are described by the ATCC, or in Freshney (1994), supra, any of which can be used. For example, murine myelomas, n51, VERO, HeT, SF9, CV-1, CHO, and other cells can be used. In some embodiments, a cell, e. g., an animal cell, that normally expresses a UCP protein can be used. For example, a brown adipose cell expressing UCP1 can be used, or a brain, muscle, or fat cell expressing UCP2 can be used.

Cells can be used at any of a wide range of densities, depending on the dye, the test agent, and the particular assay conditions. For instance, a density of about OD₆₀₀=0.01 to 1 is used, for example between about 0.05 and 0.5, e.g. about 0.1.

A large number of uncoupling proteins have been identified from numerous organisms, any of which can be used in the present invention. For example, UCP1, UCP2 (see, e. g. Fleury, et al. (1997) Nature Genetics 15: 269), UCP3, UCP4 (see, e. g., Mao et al. (1999) FEBSLett., 443: 326), BMCP1 (see, e. g., Sanchis, et al. (1998) J. Biol. Chem. 273: 34611) or homologs or derivatives thereof, can be used. UCPs have been shown to possess proton transporting activity, and to typically have six alpha-helical transmembrane domains. UCP 1-4 are homologous to each other. UCP proteins can be derived from, e. g., mammals, plants, fish, worms, insects, fungi, or any other eukaryote. Amino acid and nucleotide sequences for a multitude of UCP proteins can be found, e. g., by accessing GenBank at the National Institute of Biotechnology Information (www. ncbi. nlm. nih. gov) (see, e. g. accession numbers Y18291, NM-003356.1, AF096289, AF110532, AF036757, AF092048, and others). UCP proteins are also described, e. g., in Tartaglia (1988), U.S. Pat. No. 5,853,975, and in Science 280: 1369 (1998).

Methods for expressing heterologous proteins in cells are well known to those of skill in the art, and are described, e. g., in Ausubel (1999), Guthrie and Fink (1991), Sherman, et al. (1982) Vlethods ineast Genetics, Cold Spring Harbor Laboratories, Freshney, and others. Typically, in such embodiments, a polynucleotide encoding a heterologous protein of interest will be operably linked to an appropriate expression control sequence for the particular host cell in which the heterologous protein is to be expressed. Any of a large number of well-known promoters can be used in such method. The choice of the promoter will depend on the expression levels to be achieved and on the desired cellular specificity. Additional elements such as polyadenylation signals, 5′ and 3′ untranslated sequences, etc. are also described in well-known reference books.

In metazoan (animals having the body composed of cells differentiated into tissues and organs) cells, promoters and other elements for expressing heterologous proteins are commonly used and are well known to those of skill. See, e. g., Cruz & Patterson (1973) Tissue Culture, Academic Press; Meth. Enzymology 68 (1979), Academic Press; Freshney, 3rd Edition (1994) Culture of Animal Cells: A Manual of Basic Techniques, Wiley-Liss. Promoters and control sequences for such cells include, e. g., the commonly used early and late promoters from Simian Virus 40 (SV40), or other viral promoters such as those from polyoma, adenovirus 2, bovine papilloma virus, or avian sarcoma viruses, herpes virus family (e. g., cytomegalovirus, herpes simplex virus, or Epstein-Barr Virus), or immunoglobulin promoters and heat shock promoters (see, e. g. Sambrook, Ausubel, Meth. Enzymology Pouwells, et al., supra (1987)). In addition, regulated promoters, such as metallothionein, (i. e., MT-1 and MT- 2), glucocorticoid, or antibiotic gene “switches” can be used. Enhancer regions of such promoters can also be used.

Expression cassettes are typically introduced into a vector that facilitates entry of the expression cassette into a host cell and maintenance of the expression cassette in the host cell. Such vectors are commonly used and are well know to those of skill in the art. Numerous such vectors are commercially available, e. g., from Invitrogen, Stratagene, Clontech, etc., and are described in numerous guides, such as Ausubel, Guthrie, Strathem, or Berger, all supra. Such vectors typically include promoters, polyadenylation signals, etc. in conjunction with multiple cloning sites, as well as additional elements such as origins of replication, selectable marker genes (e. g., LEU2, URA3, TRP 1, HIS3, GFP), centromeric sequences, etc.

For expression in mammalian cells, any of a number of vectors can be used, such as pSV2, pBC12BI, and p91023, as well as lytic virus vectors (e. g., vaccinia virus, adenovirus, baculovirus), episomal virus vectors (e. g., bovine papillomavirus), and retroviral vectors (e. g., murine retroviruses).

As used herein, the term “disorder” refers to an ailment, disease, illness, clinical condition, or pathological condition.

As used herein, the term “reactive oxygen species” refers to oxygen derivatives from oxygen metabolism or the transfer of free electrons, resulting in the formation of free radicals (e. g., superoxides or hydroxyl radicals).

As used herein, the term “antioxidant” refers to compounds that neutralize the activity of reactive oxygen species or inhibit the cellular damage done by said reactive species.

As used herein, the term “pharmaceutically acceptable carrier” refers to a carrier medium that does not interfere with the effectiveness of the biological activity of the active ingredient, is chemically inert, and is not toxic to the patient to whom it is administered.

As used herein, the term “pharmaceutically acceptable derivative” refers to any homolog, analog, or fragment of an agent, e.g. identified using a method of screening of the invention, that is relatively non-toxic to the subject.

The term “therapeutic agent” refers to any molecule, compound, or treatment, that assists in the prevention or treatment of disorders, or complications of disorders.

Agents or compounds identified using a method of screening of the invention may be formulated into pharmaceutical preparations for administration to mammals for prevention or treatment of disorders. In a preferred embodiment, the mammal is a human.

Compositions comprising such an agent formulated in a compatible pharmaceutical carrier may be prepared, packaged, and labeled for treatment.

If the complex is water-soluble, then it may be formulated in an appropriate buffer, for example, phosphate buffered saline or other physiologically compatible solutions.

Alternatively, if the resulting complex has poor solubility in aqueous solvents, then it may be formulated with a non-ionic surfactant such as Tween, or polyethylene glycol. Thus, the compounds and their physiologically acceptable solvates may be formulated for administration by inhalation or insufflation (either through the mouth or the nose) or oral, buccal, parenteral, rectal administration or, in the case of tumors, directly injected into a solid tumor.

For oral administration, the pharmaceutical preparation may be in liquid form, for example, solutions, syrups or suspensions, or may be presented as a drug product for reconstitution with water or other suitable vehicle before use. Such liquid preparations may be prepared by conventional means with pharmaceutically acceptable additives such as suspending agents (e. g., sorbitol syrup, cellulose derivatives or hydrogenated edible fats); emulsifying agents (e. g., lecithin or acacia); non-aqueous vehicles (e. g., almond oil, oily esters, or fractionated vegetable oils); and preservatives (e. g., methyl or propyl-p-hydroxybenzoates or sorbic acid). The pharmaceutical compositions may take the form of, for example, tablets or capsules prepared by conventional means with pharmaceutically acceptable excipients such as binding agents (e. g., pregelatinized maize starch, polyvinyl pyrrolidone or hydroxypropyl methylcellulose); fillers (e. g., lactose, microcrystalline cellulose or calcium hydrogen phosphate); lubricants (e. g., magnesium stearate, talc or silica); disintegrants (e. g., potato starch or sodium starch glycolate); or wetting agents (e. g., sodium lauryl sulphate). The tablets may be coated by methods well-known in the art.

Preparations for oral administration may be suitably formulated to give controlled release of the active compound.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e. g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e. g., gelatin for use in an inhaler or insufflator may be formulated containing a powder mix of the compound and a suitable powder base such as lactose or starch.

The compounds may be formulated for parenteral administration by injection, e. g., by bolus injection or continuous infusion. Formulations for injection may be presented in unit dosage form, e. g., in ampoules or in multi-dose containers, with an added preservative.

The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e. g., sterile pyrogen-free water, before use.

The compounds may also be formulated in rectal compositions such as suppositories or retention enemas, e. g., containing conventional suppository bases such as cocoa butter or other glycerides.

The compounds may also be formulated as a topical application, such as a cream or lotion.

In addition to the formulations described previously, the compounds may also be formulated as a depot preparation. Such long acting formulations may be administered by implantation (for example, subcutaneously or intramuscularly) or by intramuscular injection.

Thus, for example, the compounds may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

The invention also provides kits for carrying out the therapeutic regimens of the invention. Such kits comprise in one or more containers therapeutically or prophylactically effective amounts of the compositions in pharmaceutically acceptable form.

The composition in a vial of a kit may be in the form of a pharmaceutically acceptable solution, e. g., in combination with sterile saline, dextrose solution, or buffered solution, or other pharmaceutically acceptable sterile fluid. Alternatively, the complex may be lyophilized or desiccated; in this instance, the kit optionally further comprises in a container a pharmaceutically acceptable solution (e. g., saline, dextrose solution, etc.), preferably sterile, to reconstitute the complex to form a solution for injection purposes.

In another embodiment, a kit further comprises a needle or syringe, preferably packaged in sterile form, for injecting the complex, and/or a packaged alcohol pad. Instructions are optionally included for administration of compositions by a clinician or by the patient.

A “mitochondrion” (plural “mitochondria”) is a membrane-enclosed organelle found in most eukaryotic cells These organelles range from 1-10 micrometers (μm) in size. Mitochondria generate most of the cell's supply of adenosine triphosphate (ATP), used as a source of chemical energy. In addition to supplying cellular energy, mitochondria are involved in a range of other processes, such as signaling, cellular differentiation, cell death, as well as the control of the cell cycle and cell growth. Mitochondria have been implicated in several human diseases and may play a role in the aging process. Several characteristics make mitochondria unique. The number of mitochondria in a cell varies widely by organism and tissue type. Many cells have only a single mitochondrion, whereas others can contain several thousand mitochondria. The organelle is composed of compartments that carry out specialized functions. These compartments or regions include the outer membrane, the intermembrane space, the inner membrane, and the cristae and matrix. In humans, mitochondria contain about 615 distinct types of proteins, depending on the tissue of origin. Although most of a cell's DNA is contained in the cell nucleus, the mitochondrion has its own independent genome. Further, its DNA shows substantial similarity to bacterial genomes. Mitochondria can transiently store calcium, a contributing process for the cell's homeostasis of calcium (The concentrations of free calcium in the cell can regulate an array of reactions and is important for signal transduction in the cell.). The calcium is taken up into the matrix by a calcium uniporter on the inner mitochondrial membrane. It is primarily driven by the mitochondrial membrane potential. Release of this calcium back into the cell's interior can occur via a sodium-calcium exchange protein or via “calcium-induced-calcium-release” pathways. This can initiate calcium spikes or calcium waves with large changes in the membrane potential. These can activate a series of second messenger system proteins that can coordinate processes such as neurotransmitter release in nerve cells and release of hormones in endocrine cells.

Most mitochondrial proteins are synthesized as cytosolic precursors containing “uptake peptide signals” or “mitochondrial targeting signals”. Cytosolic chaperones deliver preproteins to channel linked receptors in the mitochondrial membrane. The preprotein with presequence targeted for the mitochondria is bound by receptors and the General Import Pore (GIP) (Receptors and GIP are collectively known as Translocase of Outer Membrane or TOM) at the outer membrane. The preprotein is translocated through TOM as hairpin loops. The preprotein is transported through the intermembrane space by small TIMs (which also acts as molecular chaperones) to the TIM23 or 22 (Translocase of Inner Membrane) at the inner membrane. Within the matrix the targeting sequence is cleaved off by mtHsp70. Three mitochondrial outer membrane receptors are known: TOM20, TOM22 and TOM70. TOM70 binds to internal targeting peptides and acts as a docking point for cytosolic chaperones. TOM20 binds presequences. TOM22 binds both presequences and internal targeting peptides. The TOM channel is a cation specific high conductance channel with a molecular weight of 410 kDa and a pore diameter of 21 \dot{A}. The presequence translocase23 (TIM23) is localized to the mitochondial inner membrane and acts a pore forming protein which binds precursor proteins with its N-terminal. TIM23 acts a translocator for preproteins for the mitochondrial matrix, the inner mitochondrial membrane as well as for the intermembrane space. TIM50 is bound to TIM23 at the inner mitocondrial side and found to bind presequences. TIM44 is bound on the matrix side and found binding to mtHsp70.

The presequence translocase22 (TIM22) binds preproteins exclusively bound for the inner mitochondrial membrane. Proteins are targeted to submitochondrial compartments by multiple signals and several pathways well-known in the art. Moreover, targeting to the outer membrane, intermembrane space, and inner membrane often requires another signal sequence in addition to the matrix targeting sequence, which are also well-know by the skilled person.

Suitable “uptake peptide signals” or “mitochondrial targeting signals” for the present invention are peptides of about 12 to 80 residues in length and are usually contained within N-terminal segments (presequences). “Uptake peptide signals” or “mitochondrial targeting signals” form an amphipathic alpha helix containing a number of positive charges, very few if any negative charges, and frequent hydroxylated residues, and direct the polypeptide to mitochondria. Such “uptake peptide signals” or “mitochondrial targeting signals” are well-known in the art. See e.g. Neupert, 1997, Annual Review of Biochemistry, Vol. 66: 863-917, for review.

As used herein, a “mitochondrial dye” or “intramitochondrial dye” is a dye which is able to dye a mitochondrion. The terms “mitochondrial dye” or “intramitochondrial dye” not only refers to dye which will be within the matrix of the mitochondrion, but also refers to dyes will be taken up into submitochondrial compartments or can specifically attach any of the outer membrane, intermembrane space, and inner membrane of the mitochondrion.

“Laser scanning microscopy” is a microscopy method wherein a laser beam is focused into a small point onto a fluorescent specimen. Both reflected light and fluorescent light are detected by a photomultiplier. Reflected light is deflected by a dichroic mirror, and only fluorescent light emitted from the specimen passes through the photomultiplier. In “confocal microscopy”, out of focus information is reduced by the placement of a confocal pinhole placed in front of the photomultiplier, which allows only light from the focal plane of the laser beam to pass through. Another form of laser scanning microscopy is “multiphoton fluorescence microscopy”, which is a powerful research tool that combines the advanced optical techniques of laser scanning microscopy with long wavelength multiphoton fluorescence excitation to capture high-resolution, three-dimensional images of specimens tagged with highly specific fluorophores. “Two-photon excitation microscopy” or “2-photon microscopy” is a fluorescence imaging technique that allows imaging living tissue up to a depth of one millimeter. The two-photon excitation microscope is a special variant of the multiphoton fluorescence microscope. Two-photon excitation employs a concept first described by Maria Goppert-Mayer (1906-1972) in her 1931 doctoral dissertation, and first observed in 1962 in cesium vapor using laser excitation by Isaac Abella. The concept of two-photon excitation is based on the idea that two photons of low energy can excite a fluorophore in a quantum event, resulting in the emission of a fluorescence photon, typically at a higher energy than either of the two excitatory photons. The probability of the near-simultaneous absorption of two photons is extremely low. Therefore a high flux of excitation photons is typically required, usually a femtosecond laser. In two-photon excitation microscopy an infrared laser beam is focused through an objective lens. The Ti-sapphire laser normally used has a pulse width of approximately 100 femtoseconds and a repetition rate of about 80 MHz, allowing the high photon density and flux required for two photons absorption and is tunable across a wide range of wavelengths.

The “green fluorescent protein” (GFP) is a protein, composed of 238 amino acids (26.9 kDa), originally isolated from the jellyfish Aequorea victoria/Aequorea aequorea/Aequorea forskalea that fluoresces green when exposed to blue light. The GFP from A. victoria has a major excitation peak at a wavelength of 395 nm and a minor one at 475 nm. Its emission peak is at 509 nm which is in the lower green portion of the visible spectrum. The GFP from the sea pansy (Renilla reniformis) has a single major excitation peak at 498 nm. Due to the potential for widespread usage and the evolving needs of researchers, many different mutants of GFP have been engineered. The first major improvement was a single point mutation (S65T) reported in 1995 in Nature by Roger Tsien. This mutation dramatically improved the spectral characteristics of GFP, resulting in increased fluorescence, photostablility and a shift of the major excitation peak to 488 nm with the peak emission kept at 509 nm. The addition of the 37° C. folding efficiency (F64L) point mutant to this scaffold yielded enhanced GFP (EGFP). EGFP has an extinction coefficient (denoted ε), also known as its optical cross section of 9.13×10−21 m²/molecule, also quoted as 55,000 L/(mol·cm). Superfolder GFP, a series of mutations that allow GFP to rapidly fold and mature even when fused to poorly folding peptides, was reported in 2006. As used herein, the expression “GFP” also includes these mutants.

The “yellow fluorescent protein” (YFP) is a genetic mutant of green fluorescent protein, derived from Aequorea victoria. Its excitation peak is 514 nm and its emission peak is 527 nm.

It is to be understood that for the purpose of the present invention, the term “single-barreled” excludes multi-barrel genetically encoded GFP-based calcium indicator, for instance FRET-based calcium indicator.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

### EXAMPLES

Plasmid Construction

The cDNA encoding for the mitochondrial targeting sequence (MTS) from human cytochrome C oxidase (COX) subunit VIII (MSVLTPLLLRGLTGSARRLPVPRAKIHSL [SEQ ID NO:1]) was excised from the commercially available pDsRed2Mito vector (Clontech) using Nhel/Agel restriction sites and inserted into a neuron-specific synapsin-I (syn) promoter vector (Kugler, S., et al. (2001) Neuron-specific expression of therapeutic proteins: evaluation of different cellular promoters in recombinant adenoviral vectors. Mol Cell Neurosci. 17(1): p. 78-96). To construct the plasmids encoding the GFP-based single-barrel mitochondrial Ca²⁺ indicators mGCaMP2 and mCase16, the coding cDNAs for GCaMP2 (Tallini, Y. N., et al. (2006) Imaging cellular signals in the heart in vivo: Cardiac expression of the high-signal {\Ca} indicator GCaMP2. Proc Natl Acad Sci USA. 103(12): p. 4753-8), and Case16 (Souslova, E. A., et al. (2007) Single fluorescent protein-based Ca2+ sensors with increased dynamic range. BMC Biotechnol. 7: p. 37) were first inserted into separate syn-vectors using Bglll/Notl and Xbal/Notl, respectively. From there, the GCaMP2 and Case16 cDNAs were amplified by PCR and each inserted into the syn-vector containing the MTS cDNA sequence using Agel/Notl. The 5′ start codons (ATG) were removed and 5′ Age/restriction sites were added in addition to frame-shifting single nucleotides (G) in the process. (Primers: GCaMP2, 5′-ATT ACCGGT G CGGGGTTCTCATCATC-3′ [SEQ ID NO:2], 5′-GCGCGTAACCTTGATACTTACCTGCG-3′ [SEQ ID NO:3]; Case16, 5′-ATT ACCGGT G CGTCGTAAGTGGAATAAGAC-3′ [SEQ ID NO:4], 5′-GCGCGTAACCTTGATACTTACCTGCG-3′ [SEQ ID N0:3]). All constructs were verified by DNA sequencing as well as amplified and purified using MaxiPrep Kits (Qiagen).

Slice Culture and Transfection

Organotypic hippocampal slices were prepared from Wistar rats at postnatal day 5 as described (Stoppini, L., P. A. Buchs, and D. Muller (1991) A simple method for organotypic cultures of nervous tissue. J Neurosci Methods. 37(2): p. 173-82),in accordance with the animal care and use guidelines of the Veterinary Department Basel-Stadt. After 5-7 days in vitro (DIV), cultures were transfected with syn-mGCaMP2 or syn-mCase16 using a Helios Gene Gun (BioRad). All experiments were performed 1-2 weeks after transfection (DIV 12-21).

Electrophysiology

Hippocampal slice cultures were placed in the recording chamber of the microscope and superfused with artificial cerebrospinal fluid (ACSF) containing (in mM): 127 NaCl, 2.5 KCl, 2 CaCl₂, 1 MgCl₂, 25 NaHCO₃, 1.25 NaH₂PO₄ and 25 glucose. The solution was gassed with 95% O₂, 5% CO₂ to a pH of 7.3. To block runaway excitation during electrical stimulation, 0.01 mM 2,3-Dioxo-6-nitro-1,2,3,4-tetrahydrobenzo[f]quinoxaline-7-sulfonamide (NBXQ, Tocris) and 0.01 mM 3-((R)-2-Carboxypiperazin-4-yl)-propyl-1-phosphonic acid (R-CPP, Tocris) were always added to the standard ACSF. Current-clamp whole-cell recordings of individual CA3 neurons were performed using a MultiClamp 700 B amplifier (Axon Instruments). Recording pipettes (4 -7 MΩ) were filled with intracellular solution containing (in mM): 135 K-gluconate, 10 HEPES, 4 MgCl₂, 4 Na₂-ATP, 0.4 Na₂-GTP, 10 Na₂-phosphocreatine, 3 ascorbate and 0.3 EGTA (pH: 7.3). In some experiments, EGTA was substituted by 0.3 mM of the long-wavelength synthetic Ca²⁺ indicator X-Rhod-5F (Invitrogen). If not indicated otherwise, all experiments were performed at near physiological temperature (35±1° C.).

Two-Photon Imaging and Data Analysis

A custom-built 2-photon laser scanning microscope based on a BX51WI microscope (Olympus) and a pulsed Ti:Sapphire laser (Chameleon XR, Coherent) tuned to λ=930 nm were used. Laser intensity was controlled by an electro-optic modulator (350-80, Conoptics). Images were acquired with the open source software package ‘Scanlmage’ (Pologruto, T. A., B. L. Sabatini, and K. Svoboda (2003) ScanImage: flexible software for operating laser scanning microscopes. Biomed Eng Online. 2: p. 13), written in Matlab. Fluorescence was detected in epifluorescence (LUMPlan W-IR2 60X 0.9 NA, Olympus) and transfluorescence modes (achromatic aplanatic oil immersion condenser, 1.4 NA, Olympus) using two R3896 photomultiplier tubes (PMT, Hamamatsu) behind the objective and one R3896 PMT below the condenser. A cooled H7422P-40 PMT (Hamamatsu) was further employed in order to achieve a more sensitive and less noisy detection of the green transfluorescence. For both epi- and transfluorescence detection, 725DCXR dichroic mirrors and E700SP blocking filters were used to reflect emitted photons into secondary beamsplitters, containing 560DCXR dichroic, 525/50 (green) and 610/75 (red) band pass filters (AHF Analysentechnik).

TABLE 1
Comparison of the photophysical properties of the parent non-mitochondrial sensors used.
GCaMP2 and Case16 are brighter and show a higher single wavelength excitation dynamic range
(emission intensity change upon addition of Ca²⁺) than ratiometric-pericam.

| Parent | | Molar extinction | Quantum efficiency | Brightness | | K_{d} for |
|---|---|---|---|---|---|---|
| sensor* | Ca²⁺ | coefficient ε (λₑₓ)^{†} | Φ (λ_{abs})^{‡} | (ε × Φ) | (F/F₀)ₘₐₓ^{§} | Ca²⁺ |
| YFP-based | | | | | | |
| Ratiometric- | − | 24.1 (418) | 0.3 (511) | 7.23 | | |
| pericam | − | 4.1 (494) | n.d. | | | |
| | + | 20.5 (415) | 0.18 (517) | 3.69 | 0.5x | 1.7 μM |
| | + | 10.3 (494) | n.d. | | | |
| GFP-based | | | | | | |
| GCaMP2 | − | 11.1 (400) | n.d. | | | |
| | − | 5.2 (491) | 0.7 (511) | 3.64 | | |
| | + | 5.8 (401) | n.d. | | | |
| | + | 19 (487) | 0.93 (508) | 17.67 | 4.9x | 0.15 μM |
| Case16 | − | n.d. | n.d. | 0.52 | | |
| | + | 50 | 0.17 | 8.5 | 16.5x | 1 μM |

*Data obtained from the following publications: Ratiometric-pericam (Nagai et al., 2001), GCaMP2 (Tallini et al., 2006) and Case16 (Souslova et al., 2007).
^{†}ε is the absorbance extinction coefficient (in units of 10³ M⁻¹ cm⁻¹) at the peak absorption wavelength (λ_{abs}) in nm.
^{‡}Φ is the fluorescence quantum yield (photons absorbed/photons emitted) at the peak emission wavelength (λₑₘ) in nm.
^{§}Maximum change in fluorescence (in x-fold change) upon addition of Ca²⁺ measured at peak λ_{abs}.

### REFERENCES

1. Griesbeck O: Fluorescent proteins as sensors for cellular functions. Curr Opin Neurobiol 2004, 14:636-641.
2. Miyawaki A: Visualization of the spatial and temporal dynamics of intracellular signaling. Dev Cell 2003, 4:295-305.
3. Chudakov D M, Lukyanov S, Lukyanov K A: Fluorescent proteins as a toolkit for in vivo imaging. Trends Biotechnol 2005, 23:605-613.
4. Pologruto T A, Yasuda R, Svoboda K: Monitoring neural activity and [Ca2+] with genetically encoded Ca2+ indicators. J Neurosci 2004, 24:9572-9579.
5. Palmer A E, Tsien R Y: Measuring calcium signaling using genetically targetable fluorescent indicators. Nat Protoc 2006, 1:1057-1065.
6. Baird G S, Zacharias D A, Tsien R Y: Circular permutation and receptor insertion within green fluorescent proteins. Proc Natl Acad Sci USA 1999, 96:11241-11246.
7. Nagai T, Sawano A, Park E S, Miyawaki A: Circularly permuted green fluorescent proteins engineered to sense Ca2+. Proc Natl Acad Sci USA 2001, 98:3197-3202.
8. Nakai J, Ohkura M, Imoto K: A high signal-to-noise Ca(2+) probe composed of a single green fluorescent protein. Nat Biotechnol 2001, 19:137-141.
9. Griesbeck O, Baird G S, Campbell R E, Zacharias D A, Tsien R Y: Reducing the environmental sensitivity of yellow fluorescent protein. Mechanism and applications. J Biol Chem 2001, 276:29188-29194.
10. Belousov V V, Fradkov A F, Lukyanov K A, Staroverov D B, Shakhbazov K S, Terskikh A V, Lukyanov S: Genetically encoded fluorescent indicator for intracellular hydrogen peroxide. Nat Methods 2006, 3:281-286.
11. Kawai Y, Sato M, Umezawa Y: Single color fluorescent indicators of protein phosphorylation for multicolor imaging of intracellular signal flow dynamics. Anal Chem 2004, 76:6144-6149.
12. Tallini Y N, Ohkura M, Choi B R, Ji G, Imoto K, Doran R, Lee J, Plan P, Wilson J, Xin H B, Sanbe A, Gulick J, Mathai J, Robbins J, Salama G, Nakai J, Kotlikoff M I: Imaging cellular signals in the heart in vivo: Cardiac expression of the high-signal Ca2+ indicator GCaMP2. Proc Natl Acad Sci USA 2006, 103:4753-4758.
13. Patterson G H, Lippincott-Schwartz J: A photoactivatable GFP for selective photolabeling of proteins and cells. Science 2002, 297:1873-1877.
14. Chudakov D M, Verkhusha V V, Staroverov D B, Souslova E A, Lukyanov S, Lukyanov K A: Photoswitchable cyan fluorescent protein for protein tracking. Nat Biotechnol 2004, 22:1435-1439.

1-3. (canceled)

4. An isolated nucleic acid comprising
(i) a nucleotide sequence encoding for a single-barrel genetically encoded GFP-based calcium indicator functionally linked to a mitochondrial targeting signal, or
(ii) the nucleotide sequence complementary to the nucleotide sequence of (i).

5. The isolated nucleic acid of claim 4 wherein said mitochondrial targeting signal is MSVLTPLLLRGLTGSARRLPVPRAKIHSL (SEQ ID NO:1).

6. The isolated nucleic acid of claim 4 further comprising a promoter capable of inducing the expression of the product encoded by said nucleic acid in neurons, for instance the neuron-specific synapsin-I (syn) promoter.

7. The isolated nucleic acid of claim 4, wherein said single-barrel genetically encoded GFP-based calcium indicator is a GCaMP, Case16 and/or Case12.

8. The isolated nucleic acid of claim 4, wherein said single-barrel genetically encoded calcium indicator is GCaMP2 or Case16.

9. A recombinant vector comprising the nucleic acid of claim 4.

10. A host cell comprising the vector of claim 9.

11. A kit comprising an isolated nucleic of claim 4.

12. A method of assaying the function and/or viability of mitochondria comprising the steps of expressing an isolated nucleic according to claim 4 and assessing any calcium-dependent shift of fluorescence in the mitochondria of said cell.

13. A method of screening for agents affecting the function and/or viability of mitochondria comprising the steps of expressing an isolated nucleic according to claim 4 in a cell and assessing any calcium-dependent shift of fluorescence in the mitochondria of said cell in the presence of said agent as compared to the fluorescence in the mitochondria measured in the absence of said agent.

14. The method of claim 12 wherein the fluorescence of the single-barrel genetically encoded GFP-based calcium indicator is measured by laser scanning microscopy, e.g. 2-photon microscopy or confocal microscopy.

15. The method of claim 12 wherein the fluorescence of the single-barrel genetically encoded GFP-based calcium indicator is measured by 2-photon microscopy.
